(19) United States
(12) Patent Application Publication
(10) Pub. No.: US 2022/0319715 A1
(43) Pub. Date: Oct. 6, 2022

(54) SYSTEMS AND METHODS FOR SIMULATING IMMUNE RESPONSE

(71) Applicants: McMaster University, Hamilton (CA); University of Pennsylvania, Philadelphia, PA (US)

(72) Inventors: Thomas A. Adams, II, Dundas (CA); David Christian, Lansdale, PA (US)

(21) Appl. No.: 17/708,261

(22) Filed: Mar. 30, 2022

Related U.S. Application Data
(60) Provisional application No. 63/167,673, filed on Mar. 30, 2021.

(51) Int. Cl. G16H 50/50; A61K 35/17

(52) U.S. Cl. CPC G16H 50/50; A61K 35/17

(57) ABSTRACT

Computer-implemented systems and methods are provided for simulating immune response, including a processor operable to: determine a number of naive T-cells and a number of antigen-presenting cells within a system boundary; determine a number of bound naive T-cells within the system boundary based on the number of naive T-cells and the number of antigen-presenting cells within the system boundary; determine a number of bound primed T-cells within the system boundary based on the number of bound naive T-cells within the system boundary and the age of each bound naive T-cell; and determine a number of activated T-cells within the system boundary based on the number of bound primed T-cells within the system boundary and the age of each bound primed T-cell.

### CROSS-REFERENCE TO RELATED APPLICATIONS

This application claims the benefit of U.S. Provisional Application No. 63/167,673, filed Mar. 30, 2021, the disclosure of which is hereby incorporated by reference in its entirety.

### STATEMENT REGARDING FEDERALLY SPONSORED RESEARCH OR DEVELOPMENT

This invention was made with government support under A1125563, A1126042, A1126899 awarded by the National Institutes of Health. The government has certain rights in the invention.

### FIELD

The described embodiments relate to computer models for cell populations, and, in particular, to computer-implemented systems and methods for simulating immune response.

### BACKGROUND

The following is not an admission that anything discussed below is part of the prior art or part of the common general knowledge of a person skilled in the art.

An immune system is a host defense system that protects an organism against disease. The immune system can include various cells that detect and neutralize pathogens, such as T-cells and antigen-presenting cells presenting cognate antigen (referred to herein as antigen-presenting cells). T-cells have T-cell receptors that are disposed on the surface of the T-cell. T-cell receptors can recognize and bind to particular antigen fragments that are associated with specific pathogens. Antigen-presenting cells can process antigens and present antigen fragments at the surface of the antigen-presenting cell so that the antigen fragments can be recognized by T-cells. A T-cell can bind to an antigen-presenting cell, via a presented antigen fragment, and the T-cell can mature (i.e., change cell type), in response to the binding. The matured T-cell can undergo further differentiation and the differentiated T-cells can assist in the immune response to the pathogen associated with the antigen fragment in various ways. For example, T-cells may differentiate into cytotoxic cells that selectively kill cells associated with the pathogen.

### SUMMARY

The following introduction is provided to introduce the reader to the more detailed discussion to follow. The introduction is not intended to limit or define any claimed or as yet unclaimed invention. One or more inventions may reside in any combination or sub-combination of the elements or process steps disclosed in any part of this document including its claims and figures.

In accordance with some embodiments, there is provided a computer-implemented method for modeling immune cells. The method involves operating a processor to: determine a number of naive T-cells within a system boundary; determine a number of antigen-presenting cells within the system boundary; determine a number of bound naive T-cells within the system boundary based on the number of naive T-cells within the system boundary and the number of antigen-presenting cells within the system boundary, each bound naive T-cell corresponding to a naive T-cell that is bound to an antigen-presenting cell; determine a number of bound primed T-cells within the system boundary based on the number of bound naive T-cells within the system boundary and the age of each bound naive T-cell, each bound primed T-cell corresponding to a bound naive T-cell that has matured; and determine a number of activated T-cells within the system boundary based on the number of bound primed T-cells within the system boundary and the age of each bound primed T-cell, each activated T-cell corresponding to a bound primed T-cell that has separated from an antigen-presenting cell and divided at least once.

In some embodiments, the method may further involve operating a processor to: determine a number of naive T-cells entering the system boundary; determine a number of antigen-presenting cells entering the system boundary; determine the number of naive T-cells within the system boundary based on the number of naive T-cells entering the system boundary and a previous number of naive T-cells within the system boundary; and determine the number of antigen-presenting cells within the system boundary based on the number of antigen-presenting cells entering the system boundary and a previous number of antigen-presenting cells within the system boundary.

In some embodiments, the method may further involve operating the processor to: determine a number of bound primed T-cells leaving the system boundary; and determine the number of bound primed T-cells within the system boundary based on the number of bound primed T-cells leaving the system boundary and a previous number of bound primed T-cells within the system boundary.

In some embodiments, the method may further involve operating the processor to: determine a number of descendent activated T-cells within the system boundary based on a number of parent activated T-cells within the system boundary, each descendent activated T-cell corresponding to a parent activated T-cell that has divided once, and each parent T-cell corresponding to an activated T-cell.

In some embodiments, the descendent T-cells may include second generation T-cells and third generation T-cells, the second generation T-cells corresponding to activated T-cells that have divided once, and the third generation T-cells corresponding to activated T-cells that have divided twice; and the method may further involve operating the processor to: determine a number of second generation matured T-cells within the system boundary based on a number of first generation T-cells within the system boundary; and determine a number of third generation matured T-cells within the system boundary based on the number of second generation matured T-cells within the system boundary.

In some embodiments, the method may further involve operating the processor to: determine a number of activated T-cells leaving the system boundary; and determine the number of activated T-cells outside the system boundary based on the number of activated T-cells leaving the system boundary and a previous number of activated T-cells outside the system boundary.

In some embodiments, the number of activated T-cells leaving the system boundary may be determined based on a number of activated T-cells that have divided at least twice within the system boundary.

In some embodiments, the method may further involve operating the processor to: determine a number of activated T-cells outside the system boundary based on a previous number of activated T-cells outside the system boundary.

In some embodiments, the method may further involve operating the processor to: determine a probability of a naive T-cell binding to an antigen-presenting cell; determine a probability of a bound naive T-cell maturing to a bound primed T-cell; determine a probability of a bound primed T-cell separating from an antigen-presenting cell and dividing at least once; generate at least one random number; determine the number of bound naive T-cells within the system boundary based on the at least one random number and the probability of a bound naive T-cell maturing to a bound primed T-cell; determine the number of bound primed T-cells within the system boundary based on the at least one random number and the probability of a bound naive T-cell maturing to a bound primed T-cell; and determine the number of activated T-cells within the system boundary based on the at least one random number and the probability of a bound primed T-cell separating from an antigen-presenting cell and dividing at least once.

In some embodiments, the probability of a naive T-cell binding to an antigen-presenting cell may be determined based on the number of naive T-cells within the system boundary and the number of antigen-presenting cells within the system boundary.

In some embodiments, the method may further involve operating the processor to: generate a random number of bound naive T-cells based on a single random number and the probability of a naive T-cell binding to an antigen-presenting cell; generate a random number of bound primed T-cells for each age group of the bound naive T-cells within the system boundary based on a random number for each age group of the bound naive T-cells within the system boundary and the probability of a bound naive T-cell maturing to a bound primed T-cell; generate a random number of activated T-cells for each age group of the bound primed T-cells within the system boundary based on a random number for each age group of the bound primed T-cells within the system boundary and the probability of a bound primed T-cell separating from an antigen-presenting cell and dividing at least once; determine the number of bound naive T-cells within the system boundary based on the random number of bound naive T-cells; determine the number of bound primed T-cells within the system boundary based on the random number of bound primed T-cells for each age group of the bound naive T-cells; and determine the number of activated T-cells within the system boundary based on the random number of activated T-cells for each age group of the bound primed T-cells.

In some embodiments, the random number of bound naive T-cells, the random number of bound primed T-cells, and the random number of activated T-cells may be binomial random variables.

In some embodiments, the method may further involve operating the processor to: determine a subsequent number of naive T-cells within the system boundary; determine a subsequent number of antigen-presenting cells within the system boundary; determine a subsequent number of bound naive T-cells within the system boundary based on the subsequent number of naive T-cells within the system boundary and the subsequent number of antigen-presenting cells within the system boundary; determine a subsequent number of bound primed T-cells within the system boundary based on the subsequent number of bound naive T-cells within the system boundary and the age of each bound naive T-cell; and determine a subsequent number of activated T-cells within the system boundary based on the subsequent number of bound primed T-cells within the system boundary and the age of each bound primed T-cell.

In accordance with some embodiments, there is provided a system for modeling immune cells. The system includes a processor and a storage component communicatively coupled to the processor. The processor is operable to: determine a number of naive T-cells within a system boundary; determine a number of antigen-presenting cells within the system boundary; determine a number of bound naive T-cells within the system boundary based on the number of naive T-cells within the system boundary and the number of antigen-presenting cells within the system boundary, each bound naive T-cell corresponding to a naive T-cell that is bound to an antigen-presenting cell; determine a number of bound primed T-cells within the system boundary based on the number of bound naive T-cells within the system boundary and the age of each bound naive T-cell, each bound primed T-cell corresponding to a bound naive T-cell that has matured; and determine a number of activated T-cells within the system boundary based on the number of bound primed T-cells within the system boundary and the age of each bound primed T-cell, each activated T-cell corresponding to a bound primed T-cell that has separated from an antigen-presenting cell and divided at least once. The storage component is operable to store data corresponding to the number of naive T-cells, the number of antigen-presenting cells, the number of bound naive T-cells, the number of bound primed T-cells, and the number of activated T-cells within the system boundary.

In some embodiments, the processor may be further operable to: determine a number of naive T-cells entering the system boundary; determine a number of antigen-presenting cells entering the system boundary; determine the number of naive T-cells within the system boundary based on the number of naive T-cells entering the system boundary and a previous number of naive T-cells within the system boundary; and determine the number of antigen-presenting cells within the system boundary based on the number of antigen-presenting cells entering the system boundary and a previous number of antigen-presenting cells within the system boundary.

In some embodiments, the processor may be further operable to: determine a number of bound primed T-cells leaving the system boundary; and determine the number of bound primed T-cells within the system boundary based on the number of bound primed T-cells leaving the system boundary and a previous number of bound primed T-cells within the system boundary.

In some embodiments, the processor may be further operable to: determine a number of descendent activated T-cells within the system boundary based on a number of parent activated T-cells within the system boundary, each descendent activated T-cell corresponding to a parent activated T-cell that has divided once, and each parent T-cell corresponding to an activated T-cell.

In some embodiments, the descendent T-cells may include second generation T-cells and third generation T-cells, the second generation T-cells corresponding to activated T-cells that have divided once, and the third generation T-cells corresponding to activated T-cells that have divided twice; and the processor may be further operable to: determine a number of second generation matured T-cells within the system boundary based on a number of first generation T-cells within the system boundary; and determine a number of third generation matured T-cells within the system boundary based on the number of second generation matured T-cells within the system boundary.

In some embodiments, the processor may be further operable to: determine a number of activated T-cells leaving the system boundary; and determine the number of activated T-cells outside the system boundary based on the number of activated T-cells leaving the system boundary and a previous number of activated T-cells outside the system boundary.

In some embodiments, the number of activated T-cells leaving the system boundary may be determined based on a number of activated T-cells that have divided at least twice within the system boundary.

In some embodiments, the processor may be further operable to: determine a number of activated T-cells outside the system boundary based on a previous number of activated T-cells outside the system boundary.

In some embodiments, the processor may be further operable to determine a probability of a naive T-cell binding to an antigen-presenting cell; determine a probability of a bound naive T-cell maturing to a bound primed T-cell; determine a probability of a bound primed T-cell separating from an antigen-presenting cell and dividing at least once; generate at least one random number; determine the number of bound naive T-cells within the system boundary based on the at least one random number and the probability of a bound naive T-cell maturing to a bound primed T-cell; determine the number of bound primed T-cells within the system boundary based on the at least one random number and the probability of a bound naive T-cell maturing to a bound primed T-cell; and determine the number of activated T-cells within the system boundary based on the at least one random number and the probability of a bound primed T-cell separating from an antigen-presenting cell and dividing at least once.

In some embodiments, the probability of a naive T-cell binding to an antigen-presenting cell may be determined based on the number of naive T-cells within the system boundary and the number of antigen-presenting cells within the system boundary.

In some embodiments, the processor may be further operable to: generate a random number of bound naive T-cells based on a single random number and the probability of a naive T-cell binding to an antigen-presenting; generate a random number of bound primed T-cells for each age group of the bound naive T-cells within the system boundary based on a random number for each age group of the bound naive T-cells within the system boundary and the probability of a bound naive T-cell maturing to a bound primed T-cell; generate a random number of activated T-cells for each age group of the bound primed T-cells within the system boundary based on a random number for each age group of the bound primed T-cells within the system boundary and the probability of a bound primed T-cell separating from an antigen-presenting cell and dividing at least once; determine the number of bound naive T-cells within the system boundary based on the random number of bound naive T-cells; determine the number of bound primed T-cells within the system boundary based on the random number of bound primed T-cells for each age group of the bound naive T-cells; and determine the number of activated T-cells within the system boundary based on the random number of activated T-cells for each age group of the bound primed T-cells.

In some embodiments, the random number of bound naive T-cells, the random number of bound primed T-cells, and the random number of activated T-cells may be binomial random variables.

In some embodiments, the processor may be further operable to: determine a subsequent number of naive T-cells within the system boundary; determine a subsequent number of antigen-presenting cells within the system boundary; determine a subsequent number of bound naive T-cells within the system boundary based on the subsequent number of naive T-cells within the system boundary and the subsequent number of antigen-presenting cells within the system boundary; determine a subsequent number of bound primed T-cells within the system boundary based on the subsequent number of bound naive T-cells within the system boundary and the age of each bound naive T-cell; and determine a subsequent number of activated T-cells within the system boundary based on the subsequent number of bound primed T-cells within the system boundary and the age of each bound primed T-cell.

In accordance with some embodiments, there is provided a non-transitory computer readable medium comprising instructions executable on a processor to implement the method.

### BRIEF DESCRIPTION OF THE DRAWINGS

Several embodiments will be described in detail with reference to the drawings, in which:

FIG. 1 is a block diagram of an example system for simulating immune response, in accordance with an embodiment;

FIG. 2 is a diagram of an example immune response, in accordance with an embodiment;

FIG. 3 is a flow chart of an example method for simulating immune response, in accordance with an embodiment;

FIG. 4A is a graph of an example probability distribution of a bound naive T-cell (AP) maturing to a bound primed T-cell (BP) within a given six minute window, in accordance with an embodiment;

FIG. 4B is a graph of an example probability distribution of a bound primed T-cell (BP) separating from an antigen-presenting cell (P) and dividing at least once to form a first generation activated T-cell (C) within a given six minute window, in accordance with an embodiment;

FIG. 4C is a graph of an example probability distribution of a first (C), second (D), third (E), fourth (F), fifth (G), or sixth (H) generation activated T-cell dividing to form two descendent activated T-cells within a given six minute window, in accordance with an embodiment;

FIG. 4D is a graph of an example probability distribution of a seventh (I) or eighth and later (J) generation activated T-cell dividing to form two eighth and later (J) generation activated T-cell within a given six minute window, in accordance with an embodiment;

FIG. 5 is a diagram of an example model of immune response, in accordance with an embodiment;

FIG. 6 is a diagram of another example method for simulating immune response, in accordance with an embodiment;

FIG. 7A is a graph of cell counts of naive T-cells (A), in accordance with an embodiment;

FIG. 7B is a graph of cell counts of bound naive T-cells (AP), bound primed T-cells (BP), and antigen-presenting cells (P), in accordance with an embodiment;

FIG. 7C is a graph of cell counts of first (C), second (D), third (E), and fourth (F) generation activated T-cells, in accordance with an embodiment;

FIG. 7D is a graph of cell counts of fifth (G), sixth (H), seventh (I), and eighth and later (J) generation activated T-cells, in accordance with an embodiment;

FIG. 8A is a graph of cell counts of first generation activated T-cells (C), in accordance with an embodiment;

FIG. 8B is a graph of cell counts of fourth generation activated T-cells (F), in accordance with an embodiment; and

FIG. 8C is a graph of cell counts of eighth and later generation activated T-cells (J), in accordance of an embodiment.

The drawings, described below, are provided for purposes of illustration, and not of limitation, of the aspects and features of various examples of embodiments described herein. For simplicity and clarity of illustration, elements shown in the drawings have not necessarily been drawn to scale. The dimensions of some of the elements may be exaggerated relative to other elements for clarity. It will be appreciated that for simplicity and clarity of illustration, where considered appropriate, reference numerals may be repeated among the drawings to indicate corresponding or analogous elements or steps.

### DESCRIPTION OF EXAMPLE EMBODIMENTS

Various systems or methods will be described below to provide an example of an embodiment of the claimed subject matter. No embodiment described below limits any claimed subject matter and any claimed subject matter may cover methods or systems that differ from those described below. The claimed subject matter is not limited to systems or methods having all of the features of any one system or method described below or to features common to multiple or all of the apparatuses or methods described below. It is possible that a system or method described below is not an embodiment that is recited in any claimed subject matter. Any subject matter disclosed in a system or method described below that is not claimed in this document may be the subject matter of another protective instrument, for example, a continuing patent application, and the applicants, inventors or owners do not intend to abandon, disclaim or dedicate to the public any such subject matter by its disclosure in this document.

Furthermore, it will be appreciated that for simplicity and clarity of illustration, where considered appropriate, reference numerals may be repeated among the figures to indicate corresponding or analogous elements. In addition, numerous specific details are set forth in order to provide a thorough understanding of the embodiments described herein. However, it will be understood by those of ordinary skill in the art that the embodiments described herein may be practiced without these specific details. In other instances, well-known methods, procedures and components have not been described in detail so as not to obscure the embodiments described herein. Also, the description is not to be considered as limiting the scope of the embodiments described herein.

It should also be noted that the terms “coupled” or “coupling” as used herein can have several different meanings depending in the context in which these terms are used. For example, the terms coupled or coupling may be used to indicate that an element or device can electrically, optically, or wirelessly send data to another element or device as well as receive data from another element or device. Furthermore, the term “coupled” may indicate that two elements can be directly coupled to one another or coupled to one another through one or more intermediate elements.

It should be noted that terms of degree such as “substantially”, “about” and “approximately” as used herein mean a reasonable amount of deviation of the modified term such that the end result is not significantly changed. These terms of degree may also be construed as including a deviation of the modified term if this deviation would not negate the meaning of the term it modifies.

In addition, as used herein, the wording “and/or” is intended to represent an inclusive-or. That is, “X and/or Y” is intended to mean X or Y or both, for example. As a further example, “X, Y, and/or Z” is intended to mean X or Y or Z or any combination thereof.

Furthermore, any recitation of numerical ranges by endpoints herein includes all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about” which means a variation of up to a certain amount of the number to which reference is being made if the end result is not significantly changed.

The terms “an embodiment,” “embodiment,” “embodiments,” “the embodiment,” “the embodiments,” “one or more embodiments,” “some embodiments,” and “one embodiment” mean “one or more (but not all) embodiments of the present invention(s),” unless expressly specified otherwise.

The terms “including,” “comprising” and variations thereof mean “including but not limited to,” unless expressly specified otherwise. A listing of items does not imply that any or all of the items are mutually exclusive, unless expressly specified otherwise. The terms “a,” “an” and “the” mean “one or more,” unless expressly specified otherwise.

The example embodiments of the systems and methods described herein may be implemented as a combination of hardware or software. In some cases, the example embodiments described herein may be implemented, at least in part, by using one or more computer programs, executing on one or more programmable devices comprising at least one processing element, and a data storage element (including volatile memory, non-volatile memory, storage elements, or any combination thereof). These devices may also have at least one input device (e.g. a pushbutton keyboard, mouse, a touchscreen, and the like), and at least one output device (e.g. a display screen, a printer, a wireless radio, and the like) depending on the nature of the device. The devices may also have at least one communication device (e.g., a network interface).

It should also be noted that there may be some elements that are used to implement at least part of one of the embodiments described herein that may be implemented via software that is written in a high-level computer programming language such as object oriented programming. Accordingly, the program code may be written in C, C++or any other suitable programming language and may comprise modules or classes, as is known to those skilled in object oriented programming. Alternatively, or in addition thereto, some of these elements implemented via software may be written in assembly language, machine language or firmware as needed. In either case, the language may be a compiled or interpreted language.

At least some of these software programs may be stored on a storage media (e.g. a computer readable medium such as, but not limited to, ROM, magnetic disk, optical disc) or a device that is readable by a general or special purpose programmable device. The software program code, when read by the programmable device, configures the programmable device to operate in a new, specific and predefined manner in order to perform at least one of the methods described herein.

Furthermore, at least some of the programs associated with the systems and methods of the embodiments described herein may be capable of being distributed in a computer program product comprising a computer readable medium that bears computer usable instructions for one or more processors. The medium may be provided in various forms, including non-transitory forms such as, but not limited to, one or more diskettes, compact disks, tapes, chips, and magnetic and electronic storage.

T-cells and antigen-presenting cells play a key role in an organism's immune response to pathogens. It can be useful to track T-cells and antigen-presenting cells in order to determine the status of an immune system. For example, monitoring the presence of T-cells and antigen-presenting cells can be valuable in determining the efficacy of a vaccine. However, it can be very difficult to track T-cells and antigen-presenting cells. For example, it may be challenging and time-consuming to perform in-vivo measurements of T-cell and antigen-presenting cell populations. In the context of vaccine development, the large number of vaccine candidates, dosages, and schedules may render in-vivo measurements impractical.

Computer modeling may be used to model T-cell and antigen-presenting cell populations. Modern computers can provide sufficient processing power to simulate large cell populations that would be impractical or even impossible to physically measure. However, existing computer models often suffer from inaccuracies caused by the failure to correctly represent cellular interactions in an organism. For example, existing computer models may not accurately model cell pairing or migration. Accordingly, existing computer modeling may not be suitable for accurately simulating immune response for vaccines.

The computer-implemented systems and methods for simulating immune response disclosed herein may remedy various deficiencies of existing computer models. As described herein, the immune cell modeling systems and methods may simulate various aspects of T-cells and antigen-presenting cells that were not considered by existing computer models. For example, the simulation systems and methods can accurately predict binding between T-cells and antigen-presenting cells. Furthermore the simulation systems and methods can accurately simulate the migration of T-cell and antigen-presenting cell populations. Accordingly, the systems and methods described herein can provide improved computer modeling of immune cell populations having greater accuracy as compared to existing computer models. The high accuracy of the simulation systems and methods can thus enable computer systems to be used for vaccine development. The simulation systems and methods can therefore improve the performance of computer systems.

Although various embodiments are described herein with respect to T-cells and antigen-presenting cells, it should be appreciated that the simulation systems and methods described herein may be used to model other types of cells, in some embodiments.

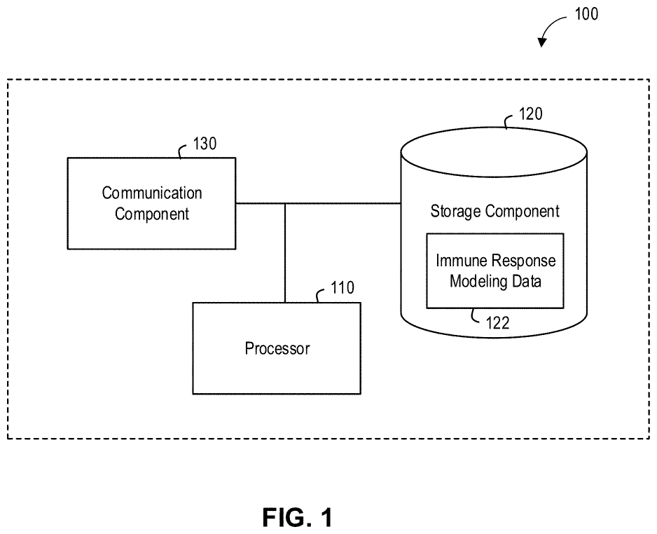

Referring now to FIG. 1, there is shown an example system 100 for simulating immune response. The simulation system 100 can be used to implement the various simulation methods described herein. The simulation system 100 can include a processor 110, a storage component 120, and a communication component 130. The processor 110, the storage component 120, and the communication component 130 and be coupled so that various data can be communicated therebetween.

The processor 110, the storage component 120, and the communication component 130 may be combined into a fewer number of components or may be separated into further components. In some embodiments, some of the components may be distributed over a wide geographic area and connected via a network. For example, the simulation system 100 may be implemented using a plurality of computer servers distributed over a wide geographic area and connected via a network. This may allow the simulation system 100 to employ parallel processing and/or distributed computing to increase processing power. The network can include any network capable of carrying data, including the Internet, Ethernet, plain old telephone service (POTS) line, public switch telephone network (PSTN), integrated services digital network (ISDN), digital subscriber line (DSL), coaxial cable, fiber optics, satellite, mobile, wireless (e.g. Wi-Fi, WiMAX), SS7 signaling network, fixed line, local area network, wide area network, and others, including any combination of these, capable of interfacing with, and enabling communication therebetween. In some embodiments, the simulation system 100 can be an electronic tablet device, a personal computer, workstation, server, portable computer, mobile device, personal digital assistant, laptop, smart phone, WAP phone, an interactive television, video display terminals, gaming consoles, and portable electronic devices or any combination of these.

The processor 110 can be implemented with any suitable processor, controller, digital signal processor, graphics processing unit, application specific integrated circuits (ASICs), and/or field programmable gate arrays (FPGAs) that can provide sufficient processing power for the configuration, purposes and requirements of the simulation system 100. The processor 110 can include more than one processor with each processor being configured to perform different dedicated tasks.

The communication component 130 can include any interface that enables the simulation system 100 to communicate with various devices and other systems. The communication component 130 can include at least one of a serial port, a parallel port or a USB port, in some embodiments. The communication component 130 may also include an interface to component via one or more of an Internet, Local Area Network (LAN), Ethernet, Firewire, modem, fiber, or digital subscriber line connection. Various combinations of these elements may be incorporated within the communication component 130. For example, the communication component 130 may receive input from various input devices, such as a mouse, a keyboard, a touch screen, a thumbwheel, a track-pad, a track-ball, a card-reader, voice recognition software and the like depending on the requirements and implementation of the simulation system 100.

The storage component 120 can include RAM, ROM, one or more hard drives, one or more flash drives or some other suitable data storage elements such as disk drives. The storage component 120 can include one or more databases for storing data 122 associated with the models simulated by the simulation system 100. In some embodiments, the storage component 120 can be used to store an operating system and programs. For instance, the operating system provides various basic operational processes for the processor 110. The programs may include various user programs so that a user can interact with the processor 110 to perform various functions such as, but not limited to, viewing and/or manipulating the stored modeling data 122, as well as retrieving and/or transmitting data.

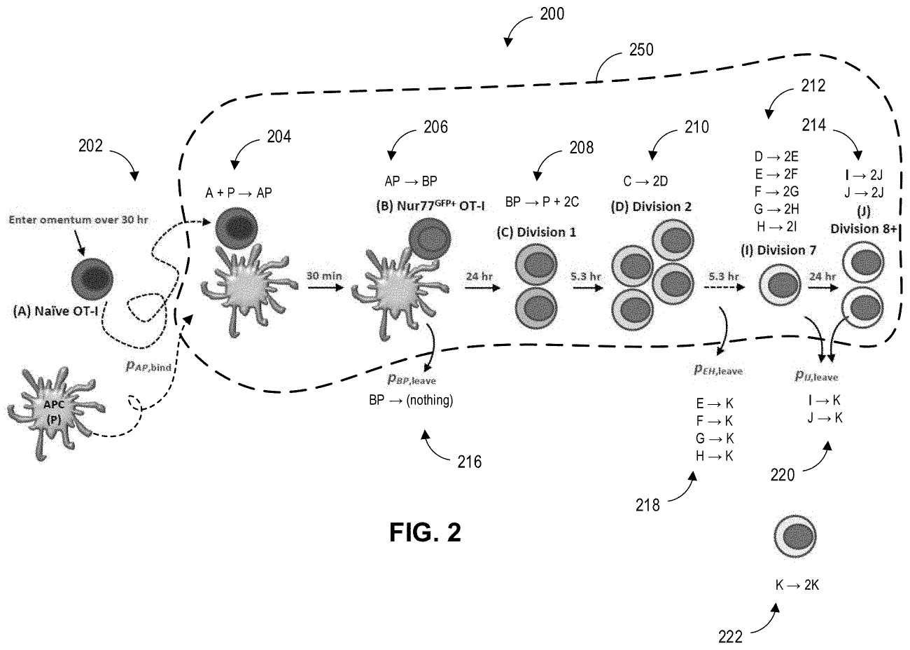

Referring now to FIG. 2, there is shown an example immune response 200 that can be modeled by the simulation system 100. As shown, the immune response 200 can involve various T-cells and cognate antigen-presenting cells. A system boundary 250 defines an area of interest of the immune response 200. The system boundary 250 can represent a portion of the body of a host organism. For example, the system boundary 250 may represent an active portion of the omentum of an organism. In other embodiments, the system boundary 250 may represent an active portion of the spleen of an organism.

At 202, naive T-cells (A) and cognate antigen-presenting cells (P) (herein referred to simply as antigen-presenting cells (P) for brevity) can enter the system boundary 250.

At 204, at least some of the naive T-cells (A) can each bind to an antigen-presenting cell (P) to form a bound naive T-cell (AP), such that:

A+P→AP

At 206, at least some of the bound naive T-cells (AP) can each mature to form a bound primed T-cell (BP), such that:

AP→BP

At 208, at least some of the bound primed T-cells (BP) can each separate from an antigen-presenting cell (P) and divide to form two first generation activated T-cells (C), such that:

BP→P+2C

At 210, at least some of the first generation activated T-cells (C) can each divide to form two second generation activated T-cells (D), such that:

C→2D

At 212, at least some of the second generation activated T-cells (D) can each divide to form two third generation activated T-cells (E); at least some of the third generation activated T-cells (E) can each divide to form two fourth generation activated T-cells (F); at least some of the fourth generation activated T-cells (F) can each divide to form two fifth generation activated T-cells (G); at least some of the fifth generation activated T-cells (G) can each divide to form two sixth generation activated T-cells (H); and at least some of the sixth generation activated T-cells (H) can each divide to form two seventh generation activated T-cells (I), such that:

D→2E

E→2F

F→2G

G→2H

H→2I

At 214, at least some of the seventh generation activated T-cells (I) can each divide to form two eighth generation activated T-cells (J), and at least some of the eighth and later generation activated T-cells (J) can each divide to form two eighth and later generation T-cells (J), such that:

I→2J

J→2J

The likelihood of a seventh (I) or eighth and later (J) generation activated T-cell dividing can be different as compared to the likelihood of a third (E), fourth (F), fifth (G), or sixth (H) generation activated T-cell dividing. In some embodiments, the likelihood of each generation dividing can be different. In other embodiments, the likelihood of one or more generations dividing can be the same as one or more other generations.

It should also be appreciated that the various simulation methods described herein be used to simulate subsequent generations (e.g., ninth, tenth, etc.) in a similar manner as the first through eight generations described herein.

At 216, at least some of the bound primed T-cells (BP) can leave the system boundary 250 (i.e., prior to separating from an antigen-presenting cell (P) and dividing to form two first generation activated T-cells (C)), such that:

BP→(nothing)

At 218, at least some of the third (E), fourth (F), fifth (G), and sixth (H) generation activated T-cells can leave the system boundary 250 to each form an activated T-cell outside the system boundary 250 (K), such that:

E→K

F→K

G→K

H→K

At 220, at least some of the seventh (I) and eighth and later (J) generation activated T-cells can leave the system boundary 250 to each form an activated T-cell outside the system boundary 250 (K), such that:

I→K

J→K

The likelihood of a seventh (I) or eighth and later (J) generation activated T-cell leaving the system boundary 250 can be different as compared to the likelihood of a third (E), fourth (F), fifth (G), or sixth (H) generation activated T-cell leaving the system boundary 250.

At 222, at least some of the activated T-cells outside the system boundary 250 (K) can each divide to form two activated T-cells outside the system boundary 250 (K) such that:

K→2K

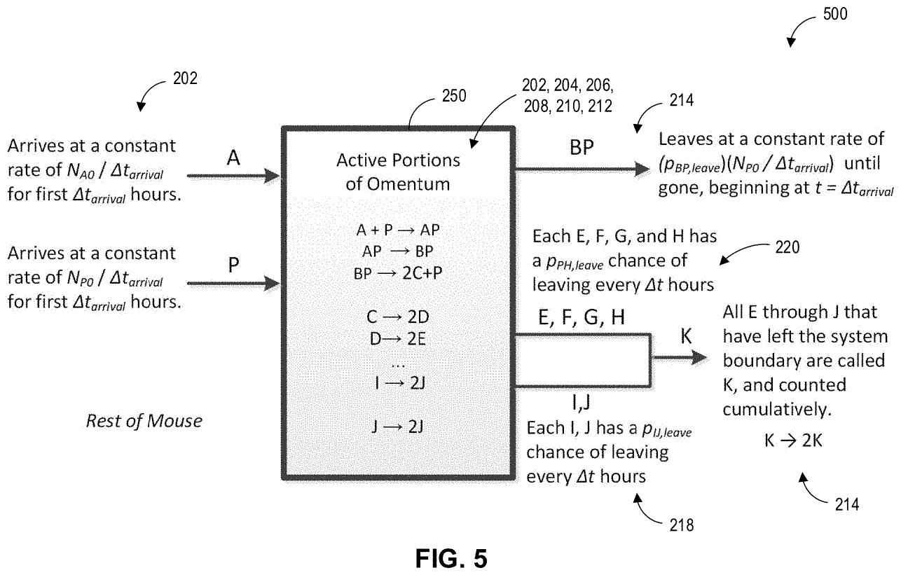

Referring now to FIG. 5, there is shown an example model 500 of the immune response 200. As shown, each process 202, 204, 206, 208, 201, 212, 214, 216, 218, 220, and 222 of the immune response 200 can be represented by a portion of the model 500. The model 500 can be implemented using the simulation system 100 to track various aspects of the immune response 200. The model 500 may be referred to as a STochastic Omentum REponse (STORE) model. The various simulation methods described herein can be implemented based on the model 500.

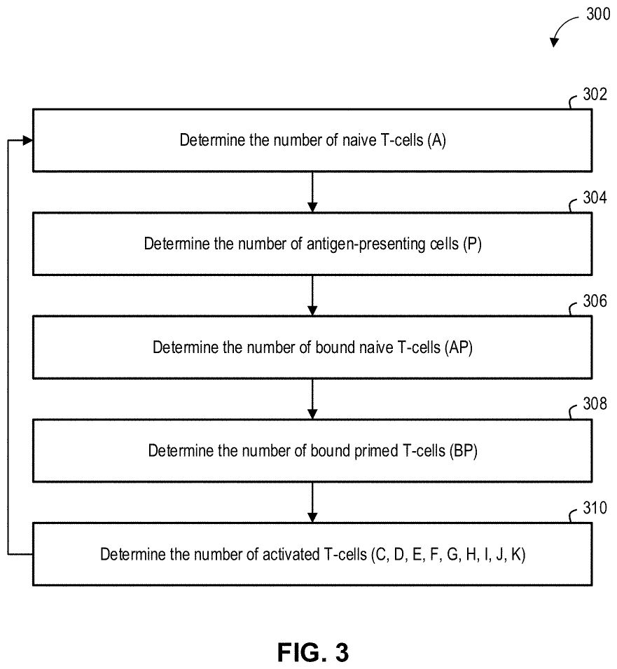

Referring now to FIG. 3, there is shown an example computer-implemented method 300 for simulating immune response. The method 300 can be used to simulate the immune response 200. The simulation method 300 can be implemented using the simulation system 100 by operating the processor 110. The simulation method 300 can be implemented based on the model 500.

At 302, the processor 110 can determine the number of naive T-cells (A) within the system boundary 250. The processor 110 can determine the number of naive T-cells (A) in various ways.

In some embodiments, the processor 110 can determine the number of naive T-cells (A) based on the number of naive T-cells (A) entering the system boundary 250 (i.e., at 202) and the previous number of naive T-cells (A) within the system boundary 250. The processor 110 can determine the number of naive T-cells (A) entering the system boundary 250 based on an arrival rate of naive T-cells (A). For example, for a linear arrival rate N_{A0}/ [symbol] tₐᵣᵣᵢᵥₐₗ, where N_{A0} naive T-cells (A) arrive during a time [symbol] tₐᵣᵣᵢᵥₐₗ, the processor 110 can determine the number of naive T-cells (A) entering the system boundary 250 beginning at a particular time t with a time step of length [symbol] t as:

$${N}_{A , thistimestep} = round\_to \_nearest \_integer ( ( t + Δ t ) \frac{{N}_{A 0}}{Δ {t}_{arrival}} )$$

Various other arrival rates can be implemented. For example, the arrival rate of naive T-cells (A) may be quadratic and the processor 110 may determine the number of naive T-cells (A) entering the system boundary 250 as:

$${N}_{A , thistimestep} = round\_to \_nearest \_integer ( {( t + Δ t )}^{2} \frac{{N}_{A 0}}{{( Δ {t}_{arrival} )}^{2}} )$$

In other embodiments, constant, Gaussian, stepwise, sawtooth, sinusoid, pulse, or other arrival rates may be implemented. In some embodiments, the arrival rates can be cyclical so that they repeat at least once, for example simulating multiple doses of a vaccine. In various embodiments, the arrivals can occur at different times, for example simulating different vaccine delivery or injection methods.

In some embodiments, the arrival rate of the naive T-cells (A) can be determined based on a predetermined value. For example, N_{A0} and [symbol] tₐᵣᵣᵢᵥₐₗ may be supplied by a user.

At 304, the processor 110 can determine the number of antigen-presenting cells (P) within the system boundary 250. The processor 110 can determine the number of antigen-presenting cells (P) in various ways.

In some embodiments, the processor 110 can determine the number of antigen-presenting cells (P) based on the number of antigen-presenting cells (P) entering the system boundary 250 (i.e., at 202) and the previous number of antigen-presenting cells (P) within the system boundary 250. The processor 110 can determine the number of the number of antigen-presenting cells (P) entering the system boundary 250 in a similar manner as for the naive T-cells (A). For example, the processor 110 may determine the number of antigen-presenting cells (P) entering the system boundary 250 N_{P,thistimestep} based on a linear arrival rate of antigen-presenting cells (P) N_{P0}/ [symbol] tₐᵣᵣᵢᵥₐₗ as:

$${N}_{P , thistimestep} = round\_to \_nearest \_integer ( ( t + Δ t ) \frac{{N}_{P 0}}{Δ {t}_{arrival}} )$$

Various arrival rates can be used. The arrival rates may be the same or different than the arrival rates for the naive T-cells (A). The arrivals can also occur at different times and may repeat.

At 306, the processor 110 can determine the number of bound naive T-cells (AP) within the system boundary 250. Each bound naive T-cell (AP) corresponds to a naive T-cell (A) that is bound to an antigen-presenting cell (P) (i.e., at 204). The processor 110 can determine the number of bound naive T-cells (AP) in various ways.

In some embodiments, the processor 110 can determine the number of bound naive T-cells (AP) based on the number of naive T-cells (A) and antigen-presenting cells

(P) within the system boundary 250. For example, the processor 110 may determine the number of bound naive T-cells (AP) based on a probability of a naive T-cell binding (A) to an antigen-presenting cell (P) and at least one random number.

The processor 110 can determine the probability of a naive T-cell binding (A) to an antigen-presenting cell (P) based on the number of naive T-cells (A) and antigen-presenting cells (P) within the system boundary. For example, processor 110 can determine the probability of a particular naive T-cell (A) within the system boundary 250 binding to any antigen-presenting cells (P) as:

$${P}_{AP} = {p}_{AP , bind} \frac{{N}_{A}}{{N}_{A} + {N}_{P}}$$

In some embodiments, p_{AP,bind} can be a predetermined value. For example, p_{AP,bind} may be supplied by a user.

In some embodiments, the processor 110 can generate a random number for each antigen-presenting cell (P) within the system boundary 250 and determine the number of bound naive T-cells (AP) based on the probability and each random number. For example, the processor 110 may generate uniformly distributed random numbers between 0 and 1, and evaluate each random number to determine whether the random number is greater than the probability. The processor 110 can then count the number of random numbers that are greater the probability to determine the number of bound naive T-cells (AP).

In other embodiments, the processor 110 can generate a single random number and generate a second random number based on the probability and the single random number. The processor 110 can then determine the number of bound naive T-cells (AP) within the system boundary 250 based on the second random number. In some embodiments, the second random number may be a binomial random variable. For example, the processor 110 may generate a random number r that is normally distributed centered on 0, and a binomial distribution having a mean μ=NₚP_{AP} and a standard deviation σ=\sqrt{NₚP_{AP}(1−P_{AP})}. The processor 110 can then determine the number of number of bound naive T-cells (AP) as:

Nₜᵣₐₙₛᵢₜᵢₒₙₛ=max(0,min(round_to_nearest_integer(σr+μ),N_{P}N_{A}))

Generating a single random number, as compared to a random number for each antigen-presenting cell (P), can require less computational effort, reducing run time while maintaining stochastic characteristics of the model. A binomial distribution may be a sufficiently accurate approximation when there are a relatively large number of antigen-presenting cells (P), such as when the number exceeds 20.

At 308, the processor 110 can determine the number of bound primed T-cells (BP) within the system boundary 250. Each bound primed T-cell (BP) corresponds to a bound naive T-cell (AP) that has matured (i.e., at 206). The processor 110 can determine the number of bound primed T-cells (BP) in various ways.

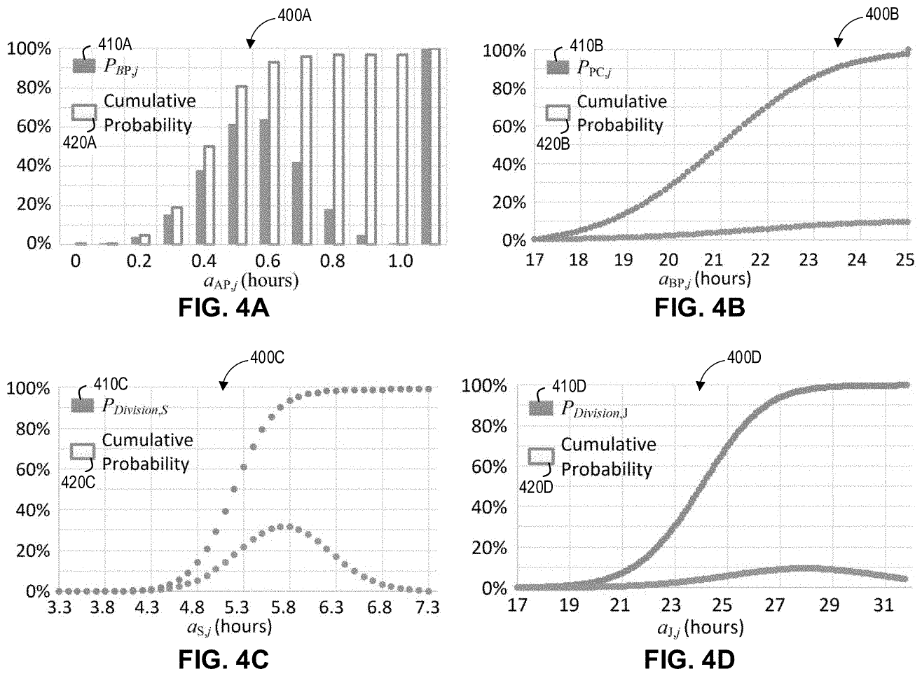

In some embodiments, the processor 110 can determine the number of bound primed T-cells (BP) based on the number of bound naive T-cells (AP) within the system boundary 250 and the age of each bound naive T-cell (AP). For example, the processor 110 may determine the number of bound primed T-cells (BP) based on the probability of a bound naive T-cell (AP) maturing to a bound primed T-cell (BP) and at least one random number. The processor can determine the probability based on the age of the bound naive T-cell (AP). For example, the probability can be determined based on a probability distribution P_{BP,j} that is dependent on age of the bound naive T-cell (AP) a_{AP,j}. For instance, FIG. 4A shows an example probability distribution 410A (and corresponding cumulative probability 420A) that illustrates the probability as function as cell age f(a_{AP,j}). In the illustrated example, the probability distribution 410A is approximately a normal distribution having a standard deviation of 0.15 hours, such that the cumulative probability 420A is 50% at 1.1 hours, and the cumulative probability 420A is 100% at 1.1 hours. Various probability distributions can be used to mimic the actual behavior of bound naive T-cells (AP), and the distributions may be partly influenced by the time step size.

In some embodiments, the processor 110 can generate a random number for each bound naive T-cell (AP) within the system boundary 250 and determine the number of bound primed T-cells (BP) based on the probability and each random number. In other embodiments, the processor 110 can generate a random number for each age group of bound naive T-cells (AP) within the system boundary 250 and generate a second random number based on each random number and the probability. Each age group of bound naive T-cell (AP) can include all of the bound naive T-cells (AP) that have the same age. The processor 110 can then determine the number of bound primed T-cells (BP) within the system boundary 250 based on the second random number. In various embodiments, the second random number may be a binomial random variable.

In some embodiments, the processor 110 can also determine the number of bound primed T-cells (BP) based on the number of bound primed T-cells (BP) leaving the system boundary 250 (i.e., at 216) and the previous number of bound primed T-cells (BP) within the system boundary 250. The processor 110 can determine the number of bound primed T-cells (BP) leaving the system boundary 250 based on a departure rate of bound primed T-cells (BP). In some embodiments, the departure rate can correspond to the arrival rate of the antigen-presenting cells (P). For example, for a linear arrival rate N_{P0}/ [symbol] tₐᵣᵣᵢᵥₐₗ, the processor 110 may determine the number of bound primed T-cells (BP) leaving the system boundary 250 as:

$${N}_{BP , leave} = min ( round\_to \_nearest \_integer ( {p}_{BP , leave} ( t - Δ {t}_{arrival} + Δ t ) \frac{{N}_{P 0}}{Δ {t}_{arrival}} ) , {N}_{BP}$$

In various embodiments, p_{BP,leave} can be a predetermined value. For example, the p_{BP,leave} may be supplied by a user.

In some embodiments, the processor 110 may determine that no bound primed T-cells (BP) have left the system boundary 250 while antigen-presenting cells (P) are entering the system boundary 250 (i.e., during [symbol] tₐᵣᵣᵢᵥₐₗ). That is, the processor 110 may determine the number of bound primed T-cells (BP) leaving the system boundary 250 only at times subsequent to [symbol] tₐᵣᵣᵢᵥₐₗ.

At 310, the processor 110 can determine the number of activated T-cells (C, D, E, F, G, H, I, J) within the system boundary 250. Each activated T-cell cell (C, D, E, F, G, H, I, J) corresponds to a bound primed T-cell (BP) that has separated from an antigen-presenting cell (P) (i.e., at 208) and divided at least once (i.e., at 208, 210, 212, and/or 214). The processor 110 can determine the number of activated T-cells (C, D, E, F, G, H, I, J) in various ways.

In some embodiments, the processor 110 can determine the number of activated T-cells (C, D, E, F, G, H, I, J) based on the number of bound primed T-cells (BP) within the system boundary 250 and the age of each bound primed T-cell (BP). For example, the processor 110 may determine the number of activated T-cells (C, D, E, F, G, H, I, J) based on the probability of a bound primed T-cell (BP) separating from an antigen-presenting cell (P) and dividing at least once and at least one random number. The processor 110 can determine the probability based on the age of the bound primed T-cell (BP). For example, the probability can be determined based on a probability distribution P_{PC,j} that is dependent on the age of the bound primed T-cell (BP) a_{BP,j}. For instance,

FIG. 4B shows an example probability distribution 410B (and corresponding cumulative probability 420B) that illustrates the probability as function as cell age f(a_{BP,j}). In the illustrated example, the probability distribution 410B is approximately a normal distribution having a standard deviation of 3 hours, such that the cumulative probability 420B is 0% before 17 hours, the cumulative probability 420B is 50% at 21 hours, and the cumulative probability 420B is 100% at 25 hours. Various probability distributions can be used to mimic the actual behavior of bound primed T-cells (BP), and the distributions may be partly influenced by the time step size.

In some embodiments, the processor 110 can generate a random number for each bound primed T-cell (BP) within the system boundary 250 and determine the number of activated T-cells (C, D, E, F, G, H, I, J) based on the probability and each random number. In other embodiments, the processor 110 can generate a random number for each age group of bound primed T-cells (BP) within the system boundary 250 and generate a second random number based on each random number and the probability. Each age group of bound primed T-cells (BP) can include all of the bound primed T-cells (BP) that have the same age. The processor 110 can then determine the number of bound primed T-cell (BP) within the system boundary 250 based on the second random number. In various embodiments, the second random number may be a binomial random variable.

The activated T-cells (C, D, E, F, G, H, I, J) can include different generations of activated T-cells. For example, the activated T-cells may include first (C), second (D), third (E), fourth (F), fifth (G), sixth (H), seventh (I), and eighth and later (J) generations. However, it should be appreciated that any number of generations can be tracked (e.g., nineth, tenth, etc.). The generation of an activated T-cell can correspond to the number of times it has divided. For example, a first generation activated T-cell (C) can correspond to zero divisions, a second generation activated T-cell (D) can correspond to one division, a third generation activated T-cell (E) can correspond to two divisions, and so forth. The second or later (D, E, F, G, H, I, J) generations can be referred to herein as descendent activated T-cells (i.e., descending from at least the first generation (C)). Each descendent activated T-cell (D, E, F, G, H, I, J) can have a corresponding parent activated T-cell (i.e., from which it divided from), and each parent T-cell can correspond to an activated T-cell (C, D, E, F, G, H, I, J).

In some embodiments, the processor 110 can also determine the number of activated T-cells based on the number of descendent activated T-cells within the system boundary 250. The processor 110 can determine the number of descendent T-cells based on the number of parent activated T-cells within the system boundary 250. For example, the processor 110 can determine the number of second generation activated T-cells (D) based on the number of first generation activated T-cells (C), the processor 110 can determine the number of third generation activated T-cells (E) based on the number of second generation activated T-cells (B), and so forth.

The processor 110 can determine the number of descendent activated T-cells based on the probability of a parent activated T-cell dividing to form two descendent activated T-cells (i.e., at 210, 212, and 214). The processor 110 can determine the probability based on the age of the parent activated T-cell. For example, the probability can be determined based on a probability distribution P_{division,S} that is dependent on the age of the bound primed T-cell (BP) a_{S,j}. For instance, FIG. 4C shows an example probability distribution 410C (and corresponding cumulative probability 420C) that illustrates the probability as function as cell age f(a_{S,j}) for first (C), second (D), third (E), fourth (F), fifth (G), and sixth (H) generation activated T-cells. In the illustrated example, the probability distribution 410C is approximately a normal distribution having a standard deviation of 0.5 hours, such that the cumulative probability 420C is 0% before 3.3 hours, the cumulative probability 420C is 50% at 5.2 hours, and the cumulative probability 420C is never 100%.

In some embodiments, the probability distribution may also be dependent on the generation of the parent activated T-cell. For example, eighth and later (J) generation activated T-cells may have a different probability distribution P_{division,J} than first (C), second (D), third (E), fourth (F), fifth (G), sixth (H), and seventh (I) generation activated T-cells, P_{division,S}. For instance, FIG. 4D shows an example probability distribution 410D (and corresponding cumulative probability 420D) for the seventh (I) and eighth and later (J) generation activated T-cells that is different than that the probability distribution 410C in FIG. 4C. In particular, the probability distribution 410D is generally “slower” , and the cumulative probability 420D is 100% at 31.8 hours. It should be appreciated that various probability distributions can be used to mimic actual real behavior of parent activated T-cells and the distributions may be partly influenced by the time step size.

The processor 110 can then generate a random number for each parent activated T-cell (i.e., and compare with the probability), or generate a single random number for each age group (i.e., to generate a random variable), to determine the number of descendent activated T-cells within the system boundary 250.

In some embodiments, the processor 110 can also determine the number of activated T-cells (C, D, E, F, G, H, I, J) based on the number of activated T-cells (C, D, E, F, G, H, I, J) leaving the system boundary 250 (i.e., at 218 and 220) and the previous number of activated T-cells (C, D, E, F, G, H, I, J) inside the system boundary 250.

The processor 110 can determine the number of activated T-cells (C, D, E, F, G, H, I, J) leaving the system boundary 250 based on the probability of an activated T-cell (C, D, E, F, G, H, I, J) leaving the system boundary 250. In various embodiments, the probability can be a predetermined value. For example, the probability may be supplied by a user. In some embodiments, the probability can depend on the generation of the activated T-cell (C, D, E, F, G, H, I, J). For example, the third (E), fourth (F), fifth (G) and sixth (H) generation activated T-cells can have a probability P_{EH,leave}, and the seventh (I) generation and eighth and later (J) generation activated T-cells can have a different probability P_{IJ,leave}, and the first (C) and second (D) generation activated T-cells can have a probability of zero. Hence, processor 110 may determine the number of activated T-cells (C, D, E, F, G, H, I, J) leaving the system boundary 250 based on the number of activated T-cells that have divided at least twice.

The processor 110 can then generate a random number for each activated T-cell (i.e., and compare with the probability), or generate a single random number for each age group (i.e., to generate a random variable), to determine the number of activated T-cells leaving the system boundary 250.

In some embodiments, the processor 110 can also determine the number of activated T-cells outside the system boundary 250 (K). For example, the processor 110 can determine the number of activated T-cells outside the system boundary 250 (K) based on the number of activated T-cells leaving the system boundary 250 and the previous number activated T-cells outside the system boundary 250 (K).

The processor 110 may also determine the number of activated T-cells outside the system boundary 250 (K) based on the previous number activated T-cells outside the system boundary 250 (K). For example, the number of activated T-cells outside the system boundary 250 (K) may be determined based on a probability of an activated T-cell outside the system boundary 250 (K) dividing into two cells (K) (i.e., at 222). In some embodiments, the probability may be the same as that of an eighth and later (J) generation activated T-cells (J) dividing, (i.e., the probability distribution 410D shown in FIG. 4D). It should be appreciated that various probability distributions can be used to represent the real behavior of activated T-cells outside the system boundary 250 (K).

The processor 110 can then generate a random number for each activated T-cell outside the system boundary 250 (K) (i.e., and compare with the probability), or generate a single random number for each age group (i.e., to generate a random variable), to determine the number of activated T-cell outside the system boundary 250 (K).

In some embodiments, the processor 110 can also determine the number of antigen-presenting cells (P) within the system boundary 250 (i.e., at 304) based on the number of bound primed T-cells (BP) that have separated from an antigen-presenting cell (P) and divided at least once to form a first generation activated T-cell cell (C) (i.e., at 216 and at 310).

Subsequent to 310, acts 302, 304, 306, 308, and 310 can be executed one or more additional times. For example, the processor 110 can determine a subsequent number of naive T-cells (A) within the system boundary 250 (i.e., at 302); determine a subsequent number of antigen-presenting cells (P) within the system boundary 250 (i.e., at 304); determine a subsequent number of bound naive T-cells (AP) within the system boundary 250 based on the subsequent number of naive T-cells (A) within the system boundary 250 and the subsequent number of antigen-presenting cells (P) within the system boundary 250 (i.e., at 306); determine a subsequent number of bound primed T-cells (BP) within the system boundary 250 based on the subsequent number of bound naive T-cells (AP) within the system boundary 250 and the age of each bound naive T-cell (AP) (i.e., at 308); and determine a subsequent number of activated T-cells (C, D, E, F, G, H, I, J) within the system boundary 250 based on the subsequent number of bound primed T-cells (BP) within the system boundary 250 and the age of each bound primed T-cell (BP) (i.e., at 310).

Each iteration of the simulation method 300 can correspond to a particular time t. Each iteration can have a time step [symbol] t. That is, each iteration can correspond to a time that is incremented by [symbol] t from the time corresponding to the previous iteration. Accordingly, the simulation method 300 can be used to track T-cells and antigen-presenting cells at various times over a time period. In some embodiments, the processor 110 can determine at least one aspect of the T-cells and/or antigen-presenting cells during a subsequent iteration (i.e., at t+ [symbol] t) based on an aspect determined during a previous iteration (i.e., at t). In some embodiments, the time step [symbol] t can be 0.1 hours (6 minutes).

It should be appreciated that the order of acts 302, 304, 306, 308, and 310 shown in the illustrated example is for ease of exposition only. In various embodiments, one or more acts may be executed in a different order than that shown in FIG. 3. For example, act 304 may be executed prior to act 302. Furthermore, one or more acts of the simulation method 300 may be executed at approximately the same time or in parallel. In some embodiments, the simulation method 300 may be executed in a reverse or backwards manner as compared to the illustrated example.

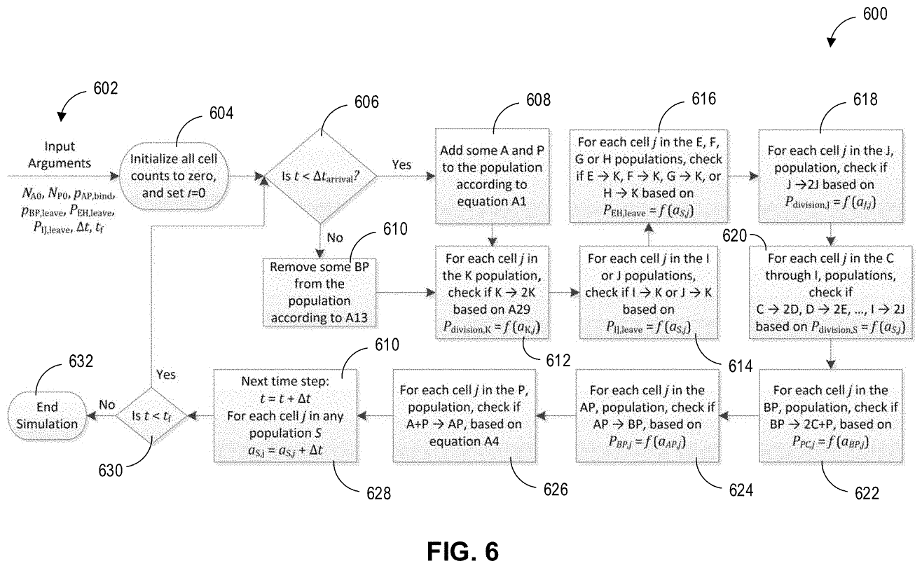

Referring to FIG. 6, there is shown another example computer-implemented method 600 for simulating immune response. Similar to the simulation method 300, the method 300 can also be used to simulate the immune response 200. The method 600 can also be implemented using the simulation system 100 by operating the processor 110. The simulation method 600 can also be implemented based on the model 500.

The simulation method 600 can be used to model a population of cells. In particular, the simulation method 600 can track the age aⱼ of each cell j and the number of cells of each cell type (A, P, AP, BP, C, D, E, F, G, H, I, J, K) (i.e., cell count) during a time period from t=0 to t=t_{f} over a plurality of time steps [symbol] t.

At 602, the processor 110 can receive input arguments. For example, a user may input the arguments via the communication component 130. The input arguments can correspond to the model 500. In some embodiments, the input arguments may include N_{A0}, N_{P0}, p_{AP,bind}, p_{BP,leave}, P_{EH,leave}, P_{IJ,leave}, [symbol] t, and/or t_{f}.

At 604, the processor 110 can initialize all cell counts to zero. The processor 110 can also set the time t to zero. In other embodiments, the cell counts may be initialized to a non-zero number.

At 606, the processor 110 can determine whether the time t is less than [symbol] ₐᵣᵣᵢᵥₐₗ. If t< [symbol] tₐᵣᵣᵢᵥₐₗ, then the simulation method will proceed to 608. Otherwise, the simulation method 600 will proceed to 610.

At 608, the processor 110 can determine whether any naive T-cells (A) and antigen-presenting cells (P) have entered the system boundary 250 (i.e., at 202). The processor 110 can then increase the cell counts for the naive T-cells (A) and the antigen-presenting cells (P) accordingly. Various arrival rates can be used to determine the number of naive T-cells (A) entering the system boundary 250. For example, the processor 110 may determine the number of naive T-cells (A) entering the system boundary 250 at time t during time step [symbol] t as:

$${N}_{A , thistimestep} = round\_to \_nearest \_integer ( ( t + Δ t ) \frac{{N}_{A 0}}{Δ {t}_{arrival}} )$$

At 610, the processor 110 can determine whether each bound primed T-cell (BP) has left the system boundary 250 (i.e., at 216). The processor 110 can then decrease the cell count for the bound primed T-cells (BP) accordingly. For example, the processor 110 may determine the number of bound primed T-cell (BP) leaving the system boundary 250 at time t during time step [symbol] t as:

$${N}_{BP , leave} = min ( round\_to \_nearest \_integer ( {p}_{BP , leave} ( t - Δ {t}_{arrival} + Δ t ) \frac{{N}_{P 0}}{Δ {t}_{arrival}} ) , {N}_{BP}$$

In the illustrated example, naive T-cells (A) and antigen-presenting cells (P) begin to entering the system boundary 250 at t=0 and continue to enter the system boundary 250 until [symbol] tₐᵣᵣᵢᵥₐₗ. Likewise, in the illustrated example, bound primed T-cells (BP) do not begin to leave the system boundary 250 until after [symbol] tₐᵣᵣᵢᵥₐₗ. However, in some embodiments, different timings may be used. That is, the naive T-cells (A) and antigen-presenting cells (P) may enter the system boundary 250 and bound primed T-cells (BP) may leave the system boundary at various different times and at different rates.

At 612, the processor 110 can determine whether each activated T-cell outside the system boundary 250 (K) has divided to form two activated T-cells outside the system boundary 250 (K) (i.e., at 222). The processor 110 can then increase the cell count for the activated T-cells outside the system boundary 250 (K) accordingly. For example, the processor 110 can determine the probability of an activated T-cell outside the system boundary 250 (K) dividing to form two activated T-cells outside the system boundary 250 (K) based on the probability distribution 410D shown in FIG. 4D. The processor 110 can then generate a random number for each activated T-cell outside the system boundary 250 (K) (i.e., and compare each random number with the probability) or generate a single random number for each age group (i.e., to generate a random variable), to determine the number of newly divided T-cells outside the system boundary 250 (K).

At 614, the processor 110 can determine whether each seventh (I) and eighth and later (J) generation activated T-cell has left the system boundary 250 to form an activated T-cell outside the system boundary 250 (K) (i.e., at 220). The processor 110 can then decrease the cell count for the seventh (I) and eighth and later (J) generation activated T-cells and increase the cell count for the activated T-cells outside the system boundary 250 (K) accordingly. For example, the processor 110 can determine the probability of a seventh (I) or eighth and later (J) generation activated T-cell leaving the system boundary 250 based on the user input P_{Ij,leave}. The processor 110 can then generate a random number for each seventh (I) and eighth and later (J) generation activated T-cells (i.e., and compare each random number with the probability) or generate a single random number for each age group (i.e., to generate a random variable), to determine the number of seventh (I) or eighth and later (J) generation activated T-cells leaving the system boundary 250.

At 616, the processor 110 can determine whether each third (E), fourth (F), fifth (G) and sixth (H) generation activated T-cell has left the system boundary 250 to form an activated T-cell outside the system boundary 250 (K) (i.e., at 218). The processor 110 can then decrease the cell count for the third (E), fourth (F), fifth (G) and sixth (H) generation activated T-cells and increase the cell count for the activated T-cells outside the system boundary 250 (K) accordingly. For example, the processor 110 can determine the probability of a third (E), fourth (F), fifth (G), or sixth (H) generation activated T-cell leaving the system boundary 250 based on the user input P_{EH,leave}. The processor 110 can then generate a random number for each third (E), fourth (F), fifth (G), and sixth (H) generation activated T-cell (i.e., and compare each random number with the probability) or generate a single random number for each age group (i.e., to generate a random variable), to determine the number of third (E), fourth (F), fifth (G), and sixth (H) generation activated T-cells leaving the system boundary 250.

At 618, the processor 110 can determine whether each eighth and later generation activated T-cell (J) has divided to form two eighth and later generation activated T-cells (J) (i.e., at 214). The processor 110 can then increase the cell count for the eighth and later generation activated T-cells (J) accordingly. For example, the processor 110 can determine the probability of an eighth and later generation activated T-cell (J) dividing based on the probability distribution 410D shown in FIG. 4D. The processor 110 can then generate a random number for each eighth and later generation activated T-cell (J) (i.e., and compare each random number with the probability) or generate a single random number for each age group (i.e., to generate a random variable), to determine the number of newly divided eighth and later generation activated T-cells (J).

At 620, the processor 110 can determine whether each first (C), second (D), third (E), fourth (F), fifth (G), sixth (H), and seventh (I) generation activated T-cell has divided to form two subsequent generation activated T-cells (i.e., at 212). The processor 110 can then increase the cell count for the respective subsequent generation activated T-cells and decrease the cell count for the respective previous generation activated T-cells accordingly. For example, the processor 110 can determine the probability of a first (C), second (D), third (E), fourth (F), fifth (G), sixth (H), or seventh (I) generation activated T-cell dividing based on the probability distribution 410C shown in FIG. 4C. The processor 110 can then generate a random number for each first (C), second (D), third (E), fourth (F), fifth (G), sixth (H), and seventh (I) generation activated T-cell (i.e., and compare each random number with the probability) or generate a single random number for each age group and generation (i.e., to generate a random variable), to determine the number of newly divided subsequent generation activated T-cells.

At 622, the processor 110 can determine whether each bound primed T-cell (BP) has separated from an antigen-presenting cell (P) and divided at least once to form two first generation activated T-cells (C) (i.e., at 208). The processor 110 can then increase the cell count for the antigen-presenting cells (P) and first generation activated T-cells (C) and decrease the cell count for the bound primed T-cells (BP). For example, the processor 110 can determine the probability of a bound primed T-cell (BP) separating from an antigen-presenting cell (P) and dividing at least once based on the probability distribution 400B shown in FIG. 4B. The processor 110 can then generate a random number for each bound primed T-cell (BP) (i.e., and compare each random number with the probability) or generate a single random number for each age group (i.e., to generate a random variable), to determine the number of newly formed first generation activated T-cells (C).

At 624, the processor 110 can determine whether each bound naive T-cell (AP) has matured to form a bound primed T-cell (BP) (i.e., at 206). The processor 110 can then increase the cell count for the bound primed T-cells (BP) and decrease the cell count for the bound naive T-cells (AP). For example, the processor 110 can determine the probability of a bound naive T-cell (AP) maturing to form a bound primed T-cell (BP) based on the probability distribution 400A shown in FIG. 4A. The processor 110 can then generate a random number for each bound naive T-cell (AP) (i.e., and compare each random number with the probability) or generate a single random number for each age group (i.e., to generate a random variable), to determine the number of newly matured bound primed T-cells (BP).

At 626, the processor 110 can determine whether each antigen-presenting cell (P) has bound to a naive T-cell (A) to form a bound naive T-cell (AP) (i.e., at 204). The processor 110 can then increase the cell count for the bound naive T-cells (AP) and decrease the cell count for the antigen-presenting cells (P) and the naive T-cells (A). For example, the processor can determine the probability of an antigen-presenting cell (P) binding to a naive T-cell (A) as:

$${P}_{AP} = {p}_{AP , bind} \frac{{N}_{A}}{{N}_{A} + {N}_{P}}$$

The processor 110 can then generate a random number for each antigen-presenting cell (P) (i.e., and compare each random number with the probability) or generate a single random number (i.e., to generate a random variable), to determine the number newly formed bound naive T-cells (AP).

At 628, the processor 110 can increase the time t by the time step [symbol] t and the age of each cell a_{S,j} by the time step [symbol] t.

At 630, the processor 110 can determine whether the time t is less than the final time for the simulation t_{f}. If t<t_{f}, then the simulation method 600 can proceed back to 606. Otherwise, the simulation method 600 can proceed to 632, at which the simulation method 600 ends.

It should be appreciated that the order of acts shown in the illustrated example is for ease of exposition only. In various embodiments, one or more acts may be executed in a different order than that shown in the illustrated example. For example, act 614 may be executed prior to act 612. Furthermore, one or more acts of the simulation method 600 may be executed at approximately the same time or in parallel. In some embodiments, the simulation method 600 may be executed in a reverse or backwards manner as compared to the illustrated example.

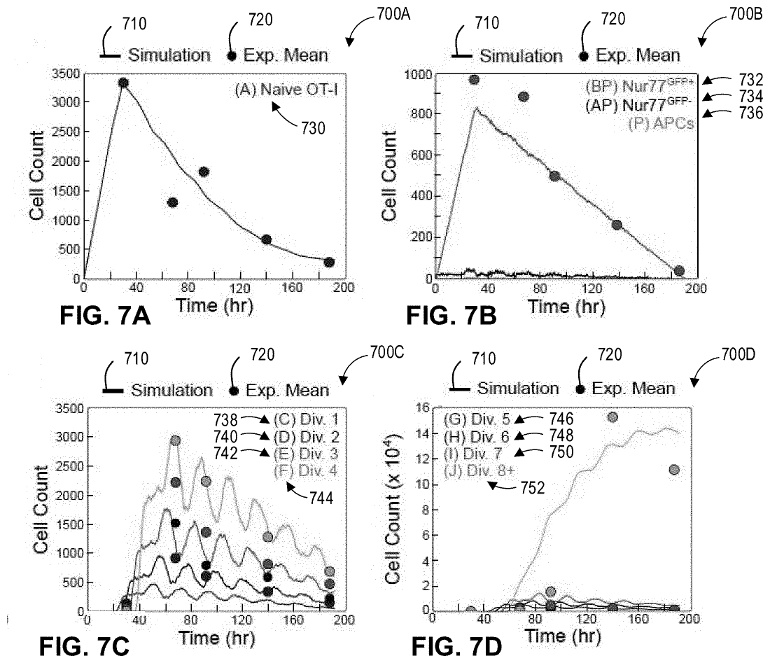

Referring now to FIGS. 7A-D, there is shown graphs 700A, 700B, 700C, and 700D illustrating various cell counts with respect to time. FIG. 7A illustrates cell counts 730 of naive T-cells (A). FIG. 7B illustrates cell counts 732 of bound naive T-cells (AP), cell counts 734 of bound primed T-cells (BP), and cell counts 736 of antigen-presenting cells (P). FIG. 7C illustrates cell counts 738 of first generation activated T-cells (C), cell counts 740 of second generation activated T-cells (D), cell counts 742 of third generation activated T-cells (E), and cell counts 744 of fourth generation activated T-cells (F). FIG. D illustrates cell counts 746 of fifth generation activated T-cells (G), cell counts 748 of sixth generation activated T-cells (H), cell counts 750 of seventh generation activated T-cells (I), and cell counts 752 of eighth generation and later activated T-cells (J).

In particular, FIGS. 7A-D illustrate each series of cell counts (i.e., corresponding to each cell type) as two subseries: a first subseries 710 of cell counts obtained from the simulation methods described herein and a second subseries 720 of cell counts obtained from experimental measurements. FIGS. 7A-D show that the cell counts 710 obtained from the simulation methods generally correspond to the cell counts 720 obtained from experimental measurements, demonstrating the accuracy of the simulation methods described herein.

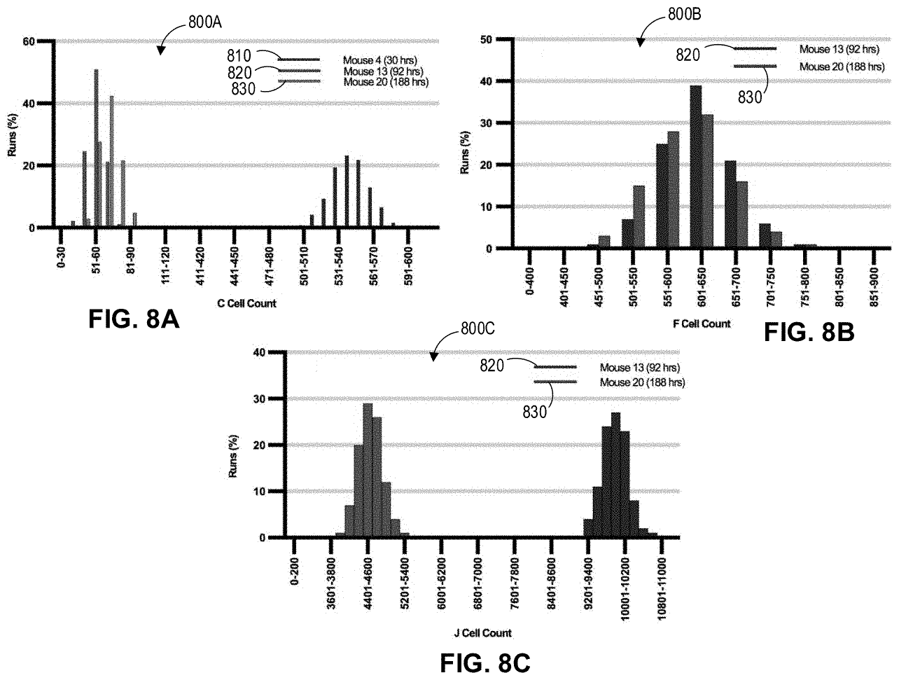

Referring now to FIGS. 8A-C, there is shown graphs 800A, 800B, and 800C illustrating cell counts for three mice 810, 820, and 830 that were obtained by running the simulation methods described herein 1000 times using the same input parameters. FIG. 8A shows cell counts of first generation activated T-cells (C). FIG. 8B shows cell counts of fourth generation activated T-cells (F). FIG. 8C shows cell counts of eighth and later generation activated T-cells (J). FIGS. 8A-C show that each cell count distribution is approximately Gaussian, demonstrating the stochastic characteristics of the simulation methods described herein.

The present invention has been described here by way of example only, while numerous specific details are set forth herein in order to provide a thorough understanding of the exemplary embodiments described herein. However, it will be understood by those of ordinary skill in the art that these embodiments may, in some cases, be practiced without these specific details. In other instances, well-known methods, procedures and components have not been described in detail so as not to obscure the description of the embodiments. Various modification and variations may be made to these exemplary embodiments without departing from the spirit and scope of the invention, which is limited only by the appended claims.

1. A computer-implemented method for simulating immune response, comprising operating a processor to:
determine a number of naive T-cells within a system boundary;
determine a number of antigen-presenting cells within the system boundary;
determine a number of bound naive T-cells within the system boundary based on the number of naive T-cells within the system boundary and the number of antigen-presenting cells within the system boundary, each bound naive T-cell corresponding to a naive T-cell that is bound to an antigen-presenting cell;
determine a number of bound primed T-cells within the system boundary based on the number of bound naive T-cells within the system boundary and the age of each bound naive T-cell, each bound primed T-cell corresponding to a bound naive T-cell that has matured; and
determine a number of activated T-cells within the system boundary based on the number of bound primed T-cells within the system boundary and the age of each bound primed T-cell, each activated T-cell corresponding to a bound primed T-cell that has separated from an antigen-presenting cell and divided at least once.

2. The method of claim 1, further comprising operating a processor to:
determine a number of naive T-cells entering the system boundary;
determine a number of antigen-presenting cells entering the system boundary;
determine the number of naive T-cells within the system boundary based on the number of naive T-cells entering the system boundary and a previous number of naive T-cells within the system boundary; and
determine the number of antigen-presenting cells within the system boundary based on the number of antigen-presenting cells entering the system boundary and a previous number of antigen-presenting cells within the system boundary.

3. The method of claim 1, further comprising operating the processor to:
determine a number of bound primed T-cells leaving the system boundary; and
determine the number of bound primed T-cells within the system boundary based on the number of bound primed T-cells leaving the system boundary and a previous number of bound primed T-cells within the system boundary.

4. The method of claim 1, further comprising operating the processor to:
determine a number of descendent activated T-cells within the system boundary based on a number of parent activated T-cells within the system boundary, each descendent activated T-cell corresponding to a parent activated T-cell that has divided once, and each parent T-cell corresponding to an activated T-cell.

5. The method of claim 4, wherein:
the descendent T-cells comprise second generation T-cells and third generation T-cells, the second generation T-cells corresponding to activated T-cells that have divided once, and the third generation T-cells corresponding to activated T-cells that have divided twice; and
the method further comprises operating the processor to:
determine a number of second generation matured T-cells within the system boundary based on a number of first generation T-cells within the system boundary; and
determine a number of third generation matured T-cells within the system boundary based on the number of second generation matured T-cells within the system boundary.

6. The method of claim 1, further comprising operating the processor to:
determine a number of activated T-cells leaving the system boundary; and
determine the number of activated T-cells outside the system boundary based on the number of activated T-cells leaving the system boundary and a previous number of activated T-cells outside the system boundary.

7. The method of claim 6, wherein:
the number of activated T-cells leaving the system boundary is determined based on a number of activated T-cells that have divided at least twice within the system boundary.

8. The method of claim 1, further comprising operating the processor to:
determine a number of activated T-cells outside the system boundary based on a previous number of activated T-cells outside the system boundary.

9. The method of claim 1, further comprising operating the processor to:
determine a probability of a naive T-cell binding to an antigen-presenting cell;
determine a probability of a bound naive T-cell maturing to a bound primed T-cell;
determine a probability of a bound primed T-cell separating from an antigen-presenting cell and dividing at least once;
generate at least one random number;
determine the number of bound naive T-cells within the system boundary based on the at least one random number and the probability of a bound naive T-cell maturing to a bound primed T-cell;
determine the number of bound primed T-cells within the system boundary based on the at least one random number and the probability of a bound naive T-cell maturing to a bound primed T-cell; and
determine the number of activated T-cells within the system boundary based on the at least one random number and the probability of a bound primed T-cell separating from an antigen-presenting cell and dividing at least once.

10. The method of claim 9, wherein:
the probability of a naive T-cell binding to an antigen-presenting cell is determined based on the number of naive T-cells within the system boundary and the number of antigen-presenting cells within the system boundary.

11. The method of claim 9, further comprising operating the processor to:
generate a random number of bound naive T-cells based on a single random number and the probability of a naive T-cell binding to an antigen-presenting;
generate a random number of bound primed T-cells for each age group of the bound naive T-cells within the system boundary based on a random number for each age group of the bound naive T-cells within the system boundary and the probability of a bound naive T-cell maturing to a bound primed T-cell;
generate a random number of activated T-cells for each age group of the bound primed T-cells within the system boundary based on a random number for each age group of the bound primed T-cells within the system boundary and the probability of a bound primed T-cell separating from an antigen-presenting cell and dividing at least once;
determine the number of bound naive T-cells within the system boundary based on the random number of bound naive T-cells;
determine the number of bound primed T-cells within the system boundary based on the random number of bound primed T-cells for each age group of the bound naive T-cells; and
determine the number of activated T-cells within the system boundary based on the random number of activated T-cells for each age group of the bound primed T-cells.

12. The method of claim 11, wherein:
the random number of bound naive T-cells, the random number of bound primed T-cells, and the random number of activated T-cells are binomial random variables.

13. The method of claim 1, further comprising operating the processor to:
determine a subsequent number of naive T-cells within the system boundary;
determine a subsequent number of antigen-presenting cells within the system boundary;
determine a subsequent number of bound naive T-cells within the system boundary based on the subsequent number of naive T-cells within the system boundary and the subsequent number of antigen-presenting cells within the system boundary;
determine a subsequent number of bound primed T-cells within the system boundary based on the subsequent number of bound naive T-cells within the system boundary and the age of each bound naive T-cell; and
determine a subsequent number of activated T-cells within the system boundary based on the subsequent number of bound primed T-cells within the system and the age of each bound primed T-cell.

14. A system for simulating immune response, comprising:
a processor operable to:
determine a number of naive T-cells within a system boundary;
determine a number of antigen-presenting cells within the system boundary;
determine a number of bound naive T-cells within the system boundary based on the number of naive T-cells within the system boundary and the number of antigen-presenting cells within the system boundary, each bound naive T-cell corresponding to a naive T-cell that is bound to an antigen-presenting cell;
determine a number of bound primed T-cells within the system boundary based on the number of bound naive T-cells within the system boundary and the age of each bound naive T-cell, each bound primed T-cell corresponding to a bound naive T-cell that has matured; and
determine a number of activated T-cells within the system boundary based on the number of bound primed T-cells within the system boundary and the age of each bound primed T-cell, each activated T-cell corresponding to a bound primed T-cell that has separated from an antigen-presenting cell and divided at least once; and
a storage component communicatively coupled to the processor and operable to store data corresponding to the number of naive T-cells, the number of antigen-presenting cells, the number of bound naive T-cells, the number of bound primed T-cells, and the number of activated T-cells within the system boundary.

15. The system of claim 14, wherein the processor is further operable to:
determine a number of naive T-cells entering the system boundary;
determine a number of antigen-presenting cells entering the system boundary;
determine the number of naive T-cells within the system boundary based on the number of naive T-cells entering the system boundary and a previous number of naive T-cells within the system boundary; and
determine the number of antigen-presenting cells within the system boundary based on the number of antigen-presenting cells entering the system boundary and a previous number of antigen-presenting cells within the system boundary.

16. The system of claim 14, wherein the processor is further operable to:
determine a number of bound primed T-cells leaving the system boundary; and
determine the number of bound primed T-cells within the system boundary based on the number of bound primed T-cells leaving the system boundary and a previous number of bound primed T-cells within the system boundary.

17. The system of claim 14, wherein the processor is further operable to:
determine a number of descendent activated T-cells within the system boundary based on a number of parent activated T-cells within the system boundary, each descendent activated T-cell corresponding to a parent activated T-cell that has divided once, and each parent T-cell corresponding to an activated T-cell.

18. The system of claim 17, wherein:
the descendent T-cells comprise second generation T-cells and third generation T-cells, the second generation T-cells corresponding to activated T-cells that have divided once, and the third generation T-cells corresponding to activated T-cells that have divided twice; and
the processor is further operable to:
determine a number of second generation matured T-cells within the system boundary based on a number of first generation T-cells within the system boundary; and
determine a number of third generation matured T-cells within the system boundary based on the number of second generation matured T-cells within the system boundary.

19. The system of claim 14, wherein the processor is further operable to:
determine a number of activated T-cells leaving the system boundary; and
determine the number of activated T-cells outside the system boundary based on the number of activated T-cells leaving the system boundary and a previous number of activated T-cells outside the system boundary.

20. The system of claim 19, wherein:
the number of activated T-cells leaving the system boundary is determined based on a number of activated T-cells that have divided at least twice within the system boundary.

21. The system of claim 14, wherein the processor is further operable to:
determine a number of activated T-cells outside the system boundary based on a previous number of activated T-cells outside the system boundary.

22. The system of claim 14, wherein the processor is further operable to:
determine a probability of a naive T-cell binding to an antigen-presenting cell;
determine a probability of a bound naive T-cell maturing to a bound primed T-cell;
determine a probability of a bound primed T-cell separating from an antigen-presenting cell and dividing at least once;
generate at least one random number;
determine the number of bound naive T-cells within the system boundary based on the at least one random number and the probability of a bound naive T-cell maturing to a bound primed T-cell;
determine the number of bound primed T-cells within the system boundary based on the at least one random number and the probability of a bound naive T-cell maturing to a bound primed T-cell; and
determine the number of activated T-cells within the system boundary based on the at least one random number and the probability of a bound primed T-cell separating from an antigen-presenting cell and dividing at least once.

23. The system of claim 22, wherein:
the probability of a naive T-cell binding to an antigen-presenting cell is determined based on the number of naive T-cells within the system boundary and the number of antigen-presenting cells within the system boundary.

24. The system of claim 22, wherein the processor is further operable to:
generate a random number of bound naive T-cells based on a single random number and the probability of a naive T-cell binding to an antigen-presenting cell;
generate a random number of bound primed T-cells for each age group of the bound naive T-cells within the system boundary based on a random number for each age group of the bound naive T-cells within the system boundary and the probability of a bound naive T-cell maturing to a bound primed T-cell;
generate a random number of activated T-cells for each age group of the bound primed T-cells within the system boundary based on a random number for each age group of the bound primed T-cells within the system boundary and the probability of a bound primed T-cell separating from an antigen-presenting cell and dividing at least once;
determine the number of bound naive T-cells within the system boundary based on the random number of bound naive T-cells;
determine the number of bound primed T-cells within the system boundary based on the random number of bound primed T-cells for each age group of the bound naive T-cells; and
determine the number of activated T-cells within the system boundary based on the random number of activated T-cells for each age group of the bound primed T-cells.

25. The system of claim 24, wherein:
the random number of bound naive T-cells, the random number of bound primed T-cells, and the random number of activated T-cells are binomial random variables.

26. The system of claim 14, wherein the processor is further operable to:
determine a subsequent number of naive T-cells within the system boundary;
determine a subsequent number of antigen-presenting cells within the system boundary;
determine a subsequent number of bound naive T-cells within the system boundary based on the subsequent number of naive T-cells within the system boundary and the subsequent number of antigen-presenting cells within the system boundary;
determine a subsequent number of bound primed T-cells within the system boundary based on the subsequent number of bound naive T-cells within the system boundary and the age of each bound naive T-cell; and
determine a subsequent number of activated T-cells within the system boundary based on the subsequent number of bound primed T-cells within the system boundary and the age of each bound primed T-cell.

27. A non-transitory computer readable medium comprising instructions executable on a processor to implement the method of claim 1.
